(19) United States
(12) Patent Application Publication
(10) Pub. No.: US 2005/0137569 A1
(43) Pub. Date: Jun. 23, 2005

(54) Activatable bioactive implantable medical device and method of use

(76) Inventors: Donald K. Jones, Lauderhill, FL (US); Juan A. Lorenzo, Davie, FL (US); Mark L. Pomeranz, Davie, FL (US); Darren Sherman, Ft. Lauderdale, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/868,152

(22) Filed: Jun. 15, 2004

Related U.S. Application Data
(63) Continuation-in-part of application No. 10/738,477, filed on Dec. 17, 2003.

(51) Int. Cl. A61M 31/00

(52) U.S. Cl. 604/500; 606/200

(57) ABSTRACT

A medial device such as a vascular occlusive device which includes a support structure and a bioactive expansible coating disposed on the support structure and an outer barrier coating which serves to prevent a reaction between the bioactive expansible coating and bodily fluids until the outer barrier is activated and removed by applying an external agent to the outer barrier thereby permitting expansion of the bioactive coating.

### CROSS-REFERENCE TO RELATED APPLICATIONS(S)

This patent application is a continuation-in-part of U.S. patent application Ser. No. 10/738,477, filed on Dec. 17, 2003, entitled, “Activatable Bioactive Implantable Medical Device And Method Of Use.”

### BACKGROUND OF INVENTION

1. Field of the Invention

The present invention relates to medical implantable devices, and more particularly, to a medical device, such as a vascular occlusive device, which includes a bioactive coating placed on the device for reacting with bodily tissue in order to promote a desired result, such as for example expansion of the bioactive coating to occlude a vessel or an aneurysm.

2. Description of the Prior Art

For many years medical devices, such as vascular occlusive devices, have been placed within the vasculature of the human body to occlude, or partially occlude, blood flow through the vasculature. Additionally, such devices have been introduced into aneurysms in order to fill, or partially fill, the aneurysm so as to reduce the pressure which is applied to the interior of the aneurysm in order to prevent further growth or expansion of the aneurysm. These devices may take the form of a coil, such as a helical coil, and are typically placed within the vessel or aneurysm by use of a delivery catheter which is inserted into the vessel and positioned such that the distal end of the delivery catheter is adjacent to a selected site for placement. Once the occlusive device is placed within a blood vessel or aneurysm, surrounding tissue reacts with the “foreign” object and begins to grow into and around the device to provide more complete occlusion of the vessel.

Examples of such delivery catheters are disclosed in U.S. Pat. No. 5,108,407, entitled “Method And Apparatus For Placement Of An Embolic Coil” and U.S. Pat. No. 5,122,136, entitled “Endovascular Electrolytically Detachable Guidewire Tip For The Electroformation Of Thrombus In Arteries, Veins, Aneurysms, Vascular Malformations And Arteriovenous Fistulas.” These patents disclose catheter systems for delivering embolic coils to preselected positions within vessels of the human body in order to treat aneurysms, or alternatively, to occlude a blood vessel at a preselected location.

Occlusive devices which take the form of coils may be helically wound coils, random wound coils, coils wound within coils or other such coil configurations. Examples of various coil configurations are disclosed in U.S. Pat. No. 5,334,210, entitled, “Vascular Occlusion Assembly” and U.S. Pat. No. 5,382,259, entitled, “Vasoocclusion Coil With Attached Tubular Woven Or Braided Fibrous Covering.” Such coils are generally formed from radiopaque metallic materials, such as platinum, gold, tungsten or alloys of these metals. Oftentimes several coils are placed at a given location within a vessel, or within an aneurysm, to more completely occlude, or partially occlude, the flow of blood through the vessel or aneurysm. Thrombus growth onto the coils further enhances the occlusive effect of the coils.

In the past, embolic coils have been placed within the distal end of a delivery catheter and when the distal end of the catheter is properly positioned, the coil may then be pushed out of the end of the catheter with, for example a guidewire, to release the coil at the desired location. This procedure of placement of the embolic coil is conducted under fluoroscopic visualization such that the movement of the coil may be monitored and the coil may be placed at a desired location.

In addition, such coils have been specifically designed to be stretch resistant, such as the vasculature occlusive coil disclosed in U.S. Pat. No. 5,853,418, entitled, “Stretch Resistant Vaso-Occlusive Coils (II)” which discloses a helically wound coil having a polymeric stretch resistant member extending through the lumen of the coil and fixedly attached to both ends of the coil to prevent the coil from stretching.

In order to increase the thrombogenicity of an embolic coil, such coils have included a coating, such as collagen, which is applied to the surface of the coil. This concept is disclosed in U.S. Pat. No. 5,690,671, entitled, “Embolic Elements And Methods And Apparatus For Their Delivery,” which discloses such a collagen coated embolic coil.

In addition, U.S. Pat. No. 5,980,550, entitled, “Water-Soluble Coating For Bioactive Vasoocclusive Devices,” discloses an embolic coil having a thrombogenic inner coating which serves as a thrombogenic agent and an outer coating of a water-soluble agent which dissolves after placement of the coil in order expose the thrombogenic inner coating to enhance the growth of thrombus into and around the coil. The water-soluble coating prevents the thrombogenic inner coating from coming into contact with the surrounding blood until the water-soluble coating is dissolved by contact with blood. While the vasculature occlusive device disclosed in this patent includes an agent for enhancing thromboginicity of the device and also includes an outer coating to prevent such activity until the outer coating is dissolved by blood flow, there is no control over when the dissolving process begins and therefore no control over the time in which the thrombogenic agent becomes activated. Without such control, it is possible that thrombus can begin forming on the coil prior to the time the coil is properly placed within a vessel, or aneurysm, therefore making it very difficult if not impossible to reposition, or remove, the improperly placed coil. Additionally, with water-soluble outer protective coatings the passive process of removing the outer coating may be so slow that the reaction may not occur in a timely manner.

Still further, U.S. Pat. No. 6,602,261, entitled, “Filamentous Embolic Device With Expansible Elements,” discloses an embolic coil having embolizing elements placed along a filament, or coil, which are comprised of a hydrophilic, polymeric, hydrogel foam material, such as hydrogel foam. After implantation of this embolic coil within an aneurysm, the water-swellable foam begins to expand and more completely fill the aneurysm. While the expansible embolizing elements of this embolic coil, upon expansion, serve to more completely fill an aneurysm, there is again no control over when the expansible elements begin to expand. With no control over the time of expansion, the embolic coils may begin expanding prior to being properly placed within an aneurysm or may expand prior to the placement of multiple coils within an aneurysm thereby making it very difficult to properly place multiple coils within the aneurysm. After the expansion of the embolizing elements has occurred, it may be very difficulty, or even impossible to reposition the embolic coil.

### SUMMARY OF THE INVENTION

In accordance with one aspect of the present invention, there is provided a medical device, such as a vascular occlusive coil, which includes a support member which may take the form of a helical coil, a bioactive expandable coating which is disposed on the support member, and an outer barrier which is disposed on the expandable coating to prevent contact between the expandable coating and a bodily fluid when the medical device is inserted into a blood vessel or an aneurysm. The expandable coating preferably takes the form of a hydrophilic, polymeric material, such as hydrogel. The outer barrier exhibits the characteristic of being inert to bodily fluid, but dissolves upon being exposed to an external agent. The external agent may take the form of a liquid medium which may be injected through a catheter into the blood vessel or aneurysm.

In accordance with another aspect of the present invention, the expansible bioactive coating takes the form of a coating of a hydrophilic material, which is applied to the support member and which serves to expand upon contact with bodily fluids, such as blood, to thereby enhance the embolizing effect of the medical device. The expandable coating preferably takes the form of a hydrophilic, polymeric material, such as hydrogel. The outer barrier takes the form of an outer coating applied to the expansible coating and prevents bodily fluid from reacting with the expansible coating until such time as the outer barrier is exposed to an external agent. The external agent may take the form of a solvent which when applied to the outer barrier through a catheter from a source outside of the body causes the outer barrier to dissolve away from the expansible coating.

In accordance with still another aspect of the present invention, there is provided a medical device, such as an vascular occlusive device, which includes an expansible element and an outer barrier applied to the expansible element which prevents a reaction between bodily fluid and the expansible element until such time as an external agent is applied to the outer barrier to thereby cause the outer barrier to dissolve away from the expansible element.

In accordance with still another aspect of the present invention, there is provided a method for treating vascular disease which includes the steps of inserting a vascular occlusion device having a support member, a bioactive expandable coating disposed on the support member, and an outer barrier disposed on the expandable coating which outer barrier exhibits the characteristic of dissolving to uncover at least a portion of the expandable coating when an external agent is applied to the outer barrier. The method includes the steps of inserting the vascular occlusive device into a blood vessel or an aneurysm and, upon election, applying an external agent through a catheter to the outer barrier to thereby cause the outer barrier to dissolve and expose at least a portion, or all, of the expandable coating to permit the expandable coating to react with bodily fluids and thereafter expand so as to partially, or completely, fill the blood vessel or aneurysm.

In accordance with still another aspect of the present invention, the method includes the steps of providing a medical device which has a bioactive expandable element which is coated with an outer barrier which exhibits the characteristic of dissolving to expose at least a portion of the expandable element when an external agent is applied to the outer barrier. This method step includes the steps of inserting the medical device into a blood vessel, and upon election, applying an external agent to the outer barrier to thereby cause the outer barrier to dissolve and expose at least a portion of the expandable element.

### BRIEF DESCRIPTION OF THE DRAWINGS

FIG. 1 is an elevational view illustrating a medical device in the form of a vascular occlusive coil in accordance with one embodiment of the present invention;

FIG. 2 is an elevational view, partly in cross-section illustrating the vascular occlusive coil as shown in FIG. 1 illustrating a bioactive expansible coating and an outer barrier coating in accordance with this embodiment of the present invention;

FIG. 3 is an elevational view, partly in cross-section illustrating the vascular occlusive coil as shown in FIG. 2 after the outer barrier coating has been removed and the expansible coating has reacted to bodily fluid and has expanded, and,

FIGS. 4A through 4C illustrate the method steps of applying multiple vascular occlusive coils as shown in FIG. 1 into an aneurysm and thereafter applying an external agent to thereby activate the embolic coils.

### DESCRIPTION OF THE PREFERRED EMBODIMENT

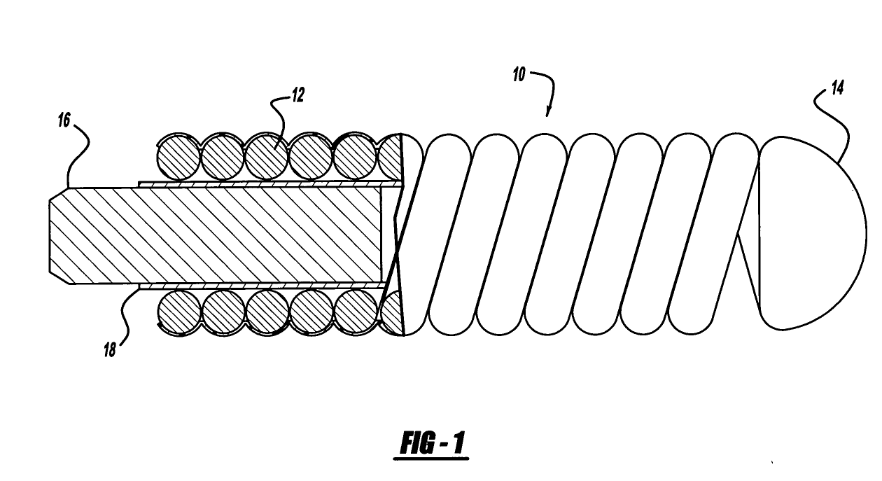

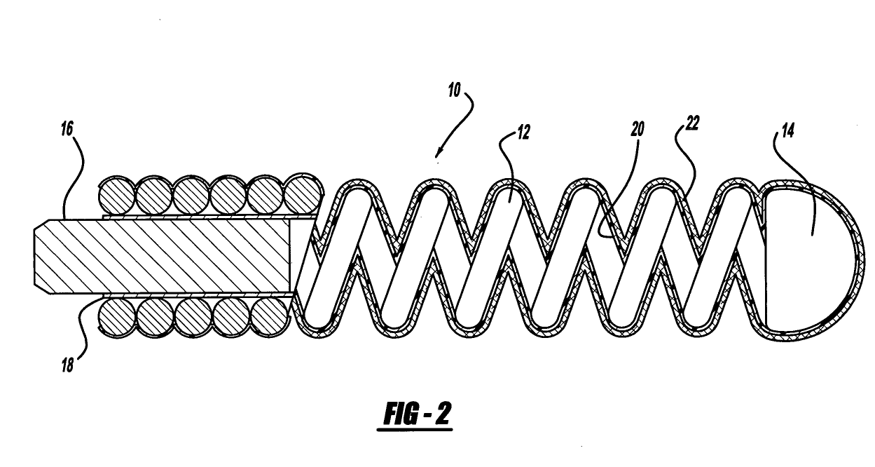

FIGS. 1 and 2 illustrate a preferred embodiment of a medical device such as an embolic coil 10 which may be placed along with other similar coils into a blood vessel or into an aneurysm in order to partially fill the aneurysm. More particularly, the embolic coil 10, or vascular occlusive coil, is a typical embolic coil which comprises a helically wound coil 12 formed from a platinum alloy wire wound into a helical configuration. The diameter of the wire is generally in the range of about 0.0007 inches to about 0.008 inches and the outer diameter of the coil 12 is preferably in a range of about 0.003 inches to about 0.055 inches. While the particular embolic coil 10 illustrated in FIGS. 1 and 2 is shown as being a straight, helically wound coil, it should be appreciated that embolic coils are formed in various configurations and may take the form of a helical wire wound in a helical configuration, a random shaped configuration or even a coil within a coil.

Preferably the embolic coil 10 includes a weld bead 14 which is attached to the distal end of the coil for providing a less traumatic distal end for the embolic coil 10. In addition, the embolic coil 10 includes a cylindrical headpiece 16 which is placed into the lumen of the helically wound coil 12 at the proximal end of the coil and is held in place by an adhesive material 18 interposed between the cylindrical headpiece 16 and the helically wound coil 12. The construction of the embolic coil 10 and an associated hydraulic deployment system for placing the embolic coil within an aneurysm is disclosed in more detail in U.S. patent application Ser. No. 10/102,154, entitled, “Small Diameter Embolic Coil Hydraulic Deployment System,” filed Mar. 19, 2002, assigned to the same assignee of the present invention and is hereby incorporated by reference.

FIG. 2 illustrates in more detail the embolic coil 10 which comprises the helically wound coil 12, a bioactive expansible coating 20 disposed upon the coil 12, and an outer barrier 22 disposed upon the expansible coating 20 for preventing the activation of the expansible coating until such time as an election is made to activate the coating. More particularly, the expansible coating 20, may take the form of a hydrophilic, polymeric material, such as a hydrogel or hydrogel foam material, which when exposed to bodily fluids expands. While the expansible coating may take the form of a hydrophilic material, it should be understood that it may take any form of any material which would expand upon reaction with bodily fluids or to other agents.

The outer barrier 22 takes the form of a coating which is disposed upon the bioactive expansible coating 20 and serves to insulate the expansible coating from adjacent bodily fluid until such time as a decision is made by a physician to activate the outer barrier 22 by applying an external agent to the barrier. The outer barrier 22 takes the form of a material which is inert to bodily fluid, but which dissolves and exposes the expansible coating 20 when the outer barrier 22 is subjected to an external agent.

In a preferred embodiment, the outer barrier 22 is comprised of ethylene vinyl alcohol, and the external agent for dissolving the outer barrier 22 is comprised of dimethyl sulfoxide (DMSO) which when applied through a catheter from an external source serves to dissolve the outer barrier 22 to thereby expose the expansible coating 20. The expansible coating 20 is comprised of a hydrophilic hydrogel or hydrogel foam material, and in particular, a water-expansible foam matrix polymer of the type disclosed in U.S. Pat. No. 5,750,585 entitled, “Super Absorbent Hydrogel Foams,” which disclosure is incorporated herein by reference. It should be appreciated that there are numerous materials which would serve as an expansible element coating, an outer barrier and an agent for dissolving or removing the outer barrier.

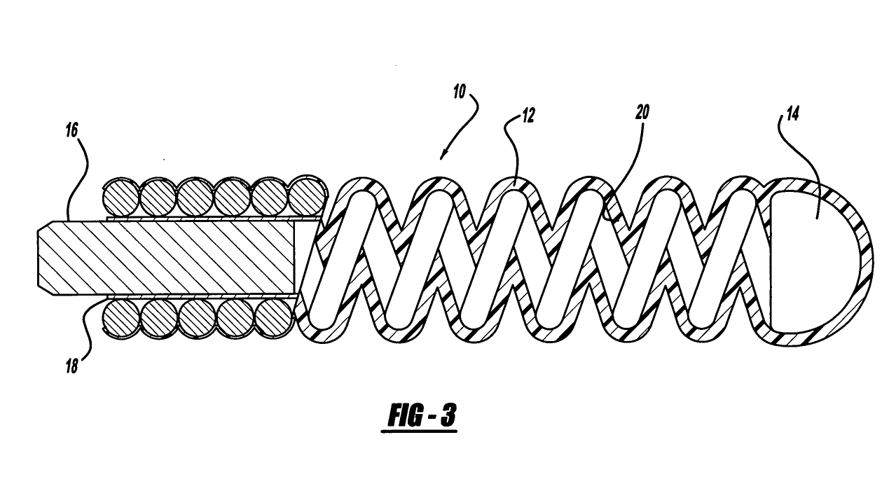

FIG. 3 illustrates in more detail the embolic coil 10 after placement into an aneurysm and after the outer barrier 22 has been dissolved thereby exposing the expansible coating 20 to bodily fluid, such as blood. As may be noted the expansible coating 20 has expanded substantially thereby causing the embolic coil 10 to more completely fill a blood vessel or an aneurysm. The outer barrier is inert to bodily fluids or blood, i.e., is not water-soluble, and the external agent, or solvent, is applied through a catheter from a source outside of the body to thereby dissolve or remove the outer barrier 22 for activation of the expansible coating 20.

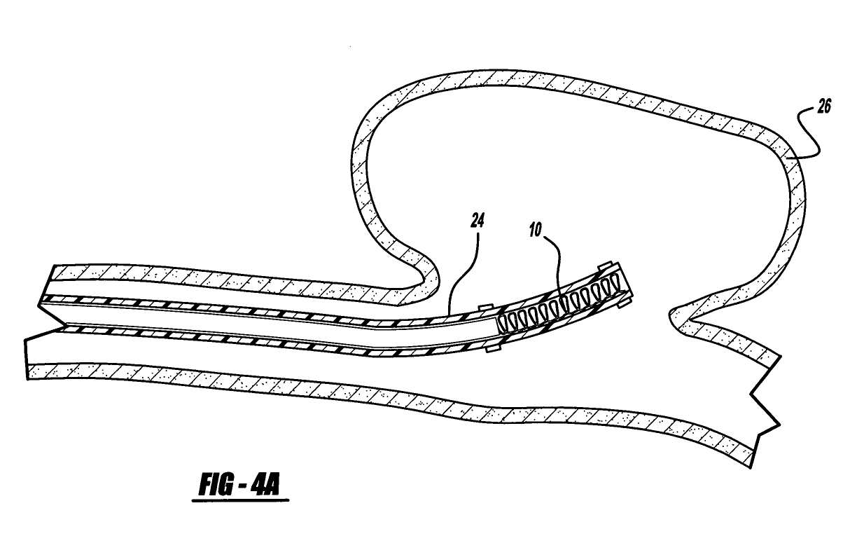

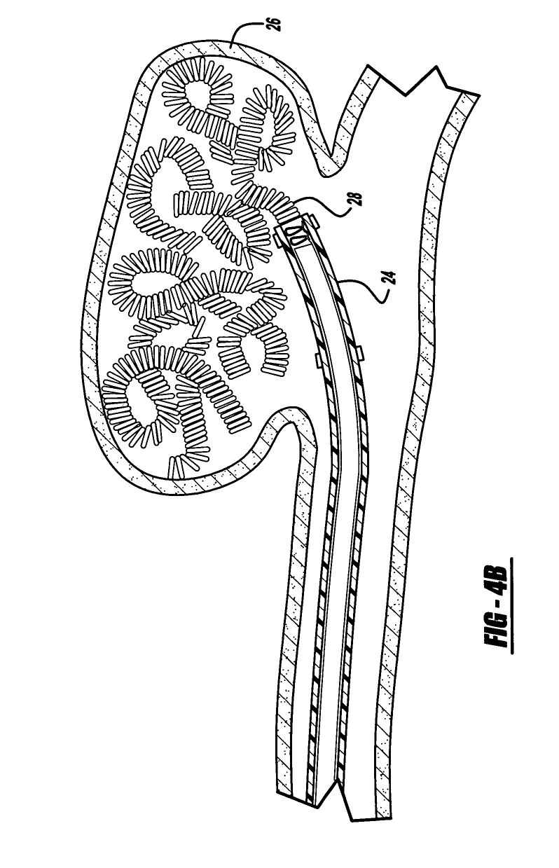

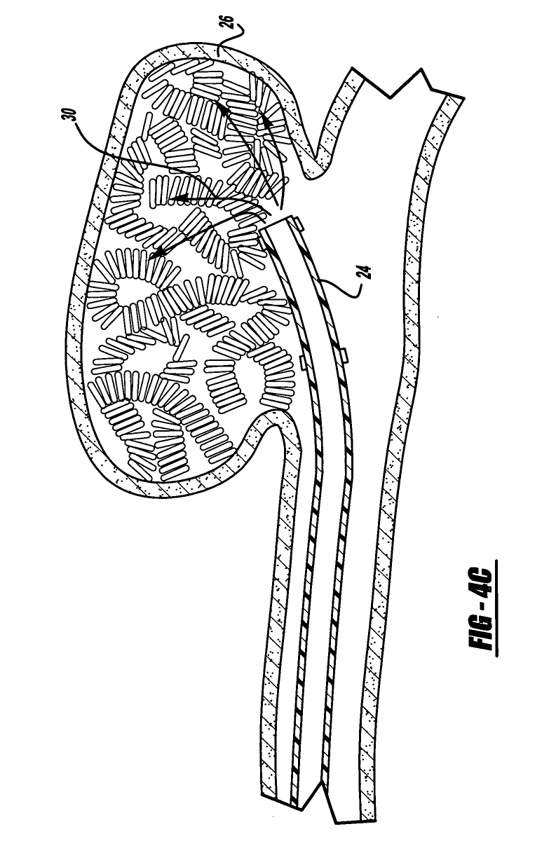

FIGS. 4A through 4C generally illustrate a method of utilizing the present invention. More particularly, FIG. 4A illustrates a delivery catheter 24 having an embolic coil 10 placed in the distal end of the catheter for delivery into an aneurysm 26. FIG. 4B illustrates the delivery catheter 24 being used to position multiple vascular occlusive coils including a final coil 28 into the aneurysm 26. FIG. 4C illustrates the application of an external agent 30, which may take the form of a solvent, for dissolving the outer barrier 22, through a catheter from a source external of the body to thereby activate the expansible coating 20. The expansible coatings 20 are illustrated in their expanded configurations.

It may be desirable to place all of the vascular occlusive coils into the aneurysm 26 prior to applying the external agent 30, however, another approach is that of placing a single coil into the aneurysm and thereafter activating that single coil, placing a second coil into the aneurysm and thereafter activating the second coil and so forth until all of the coils have been properly placed into the aneurysm. As may be appreciated, the advantage of the subject invention over prior devices is that the physician may determine at what point in time during the process of “filling” a blood vessel or an aneurysm the physician elects to activate the coil or coils for expansion.

Although a preferred embodiment of the present invention has been described, it is to be understood that various modifications may be made by those skilled in the art without departing from the scope of the claims which follow.

1. A vascular occlusion device comprising:
an expandable embolizing element; and,
an outer barrier disposed on said expandable embolizing element to prevent exposure of said embolizing element to bodily fluid when said vascular occlusion device is inserted into a blood vessel, said outer barrier exhibiting the characteristic of being non-water soluble but dissolving when an external activating agent is applied to said outer barrier.

2. A vascular occlusion device as defined in claim 1, wherein said expandable embolizing element is comprised of a hydrophilic material.

3. A vascular occlusion device as defined in claim 2, wherein said expandable embolizing element is comprised of hydrogel.

4. A vascular occlusion device as defined in claim 3, wherein the outer barrier takes the form of a coating applied to the expandable embolizing element.

5. A vascular occlusion device as defined in claim 4, wherein said outer barrier is comprised of ethylene vinyl alcohol.

6. A vascular occlusion device as defined in claim 5, wherein said external activating agent is comprised of dimethyl sulfoxide.

7. A vascular occlusion device comprising:
a support member;
an expandable embolizing element disposed on said support member; and,
an outer barrier disposed on said expandable embolizing element to prevent contact between said embolizing element and bodily fluid when said vascular occlusion device is inserted into the body, said outer barrier exhibiting the characteristic of being substantially inert to blood but dissolving and exposing a portion of said expandable embolizing element when in the presence of an external agent.

8. A vascular occlusion device as defined in claim 7, wherein said expandable embolizing element is comprised of a hydrophilic material.

9. A vascular occlusion device as defined in claim 8, wherein said expandable embolizing element is comprised of hydrogel.

10. A vascular occlusion device as defined in claim 9, wherein said outer barrier is comprised of ethylene vinyl alcohol.

11. A vascular occlusion device comprising:
an expandable embolizing element; and,
an outer barrier comprising an activatable agent, said outer barrier covering said expandable embolizing element and being inert to bodily fluid and exhibiting the characteristics of substantially preventing a reaction between the embolizing element and bodily fluid when said vascular occlusion device is inserted into the body and permitting a reaction between the embolizing element and bodily fluid upon activation by an external agent.

12. A vascular occlusion device as defined in claim 11, wherein said expandable embolizing element comprises a hydrophilic material.

13. A vascular occlusion device as defined in claim 12, wherein said expandable embolizing element is comprised of hydrogel.

14. A vascular occlusion device as defined in claim 13, wherein said outer barrier is comprised of ethylene vinyl alcohol.

15. A vascular occlusion device comprising:
a bioactive support member which when placed within the body expands when exposed to bodily fluid; and,
a barrier for preventing a reaction between the bioactive support member and bodily fluid when said vascular occlusion device is inserted into the body, said barrier exhibiting the characteristic of being inert to blood but exposing the bioactive support member to bodily fluid when an activating agent is applied to said barrier.

16. A vascular occlusion device as defined in claim 15, wherein said bioactive support member is comprised of a hydrophilic material.

17. A vascular occlusion device as defined in claim 16, wherein said bioactive support member is comprised of hydrogel.

18. A vascular occlusion device as defined in claim 17, wherein said barrier is comprised of ethylene vinyl alcohol.

19. A medical device comprising:
a support member;
an expandable embolizing element disposed on said support member; and,
an outer barrier disposed on said expandable embolizing element to prevent exposure of said embolizing element to bodily fluid when said medical device is inserted into a blood vessel, said outer barrier exhibiting the characteristic of being substantially inert to bodily fluid but dissolving when exposed to an external agent.

20. A medical device as defined in claim 19, wherein said expandable embolizing element takes the form of a coating applied to the support member.

21. A medical device as defined in claim 20, wherein said expandable embolizing element is comprised of a hydrophilic material.

22. A medical device as defined in claim 21, wherein said expandable embolizing element is comprised of a hydrogel material.

23. A medical device as defined in claim 22, wherein the outer barrier takes the form of a coating applied to the expandable embolizing element.

24. A medical device as defined in claim 23, wherein said outer barrier is comprised of ethylene vinyl alcohol.

25. A medical device as defined in claim 24, wherein said external agent is comprised of dimethyl sulfoxide.

26. A method of treatment comprising the steps of:
providing a vascular occlusion device comprising a support member, a bioactive expandable coating disposed on said support member, and an outer barrier exhibiting the characteristic of normally preventing a reaction between the expandable coating and bodily fluid and of exposing said expandable coating when an external agent is applied to said barrier;
inserting a delivery catheter into a blood vessel;
advancing the distal tip of the delivery catheter through the blood vessel until the distal tip is adjacent to a selected site within the blood vessel;
delivering said vascular occlusion device with the delivery catheter at the selected site; and,
applying said external agent through the delivery catheter and into the blood vessel to thereby activate said outer barrier to expose said bioactive expandable coating to bodily fluid to cause a reaction between the expandable coating and the bodily fluid which in turn causes the expandable coating to expand within the blood vessel.

27. A method of treatment comprising the steps of:
providing a vascular occlusion device comprising an expandable embolizing support member which reacts with bodily fluid and having an outer barrier which exhibits the characteristic of normally inhibiting a reaction between said expandable support member and bodily fluid;
inserting a delivery catheter into a blood vessel;
advancing the distal tip of the delivery catheter through the blood vessel until the distal tip is adjacent a selected site in proximity to an aneurysm;
delivering said vascular occlusion device with the delivery catheter at the selected site; and,
applying an external agent through the delivery catheter to the selected site to thereby activate said outer barrier and thus expose said expandable support member to bodily fluid to cause a reaction between the expandable support member and the bodily fluid to thereby cause expansion of said expandable support member.
